(19) United States
(12) Patent Application Publication
(10) Pub. No.: US 2016/0089365 A1
(43) Pub. Date: Mar. 31, 2016

(54) IMMUNOSTIMULATORY COMPOUND

(71) Applicant: Eisai R&D Management Co., Ltd., Tokyo (JP)

(72) Inventors: Yu Kato, Tsukuba (JP); Sho Tachino, Tsukuba (JP); Takayuki Nakagawa, Tsukuba (JP); Kimiyo Tabata, Tsukuba (JP); Kiyoshi Okamoto, Tsukuba (JP); Yusaku Hori, Tsukuba (JP)

(21) Appl. No.: 14/862,349

(22) Filed: Sep. 23, 2015

Related U.S. Application Data
(60) Provisional application No. 62/055,239, filed on Sep. 25, 2014.

(51) Int. Cl. A61K 31/47

(52) U.S. Cl. CPC A61K 31/47

(57) ABSTRACT

An immunostimulatory compound is disclosed.

### CROSS-REFERENCE TO RELATED APPLICATION

This application claims priority to Provisional Application No. 62/055,239 filed on Sep. 25, 2014, which is hereby incorporated by reference in its entirety.

### TECHNICAL FIELD

The present invention generally relates to an immunostimulant. The present invention provides an immunostimulant comprising 4-(3-chloro-4-(cyclopropylaminocarbonyl)aminophenoxy)-7-methoxy-6-quinolinecarboxamide (also known as lenvatinib) or a pharmacologically acceptable salt thereof (e.g., lenvatinib mesilate).

### BACKGROUND

Kinase inhibitor is regarded as a major class of antitumor agents, and a number of kinase inhibitors have been developed so far. For example, a group of compounds having inhibitory activity against receptor tyrosine kinases, such as vascular endothelial growth factor receptor (VEGFR), are known to inhibit angiogenesis, which leads to the shrinkage of tumor.

However, there remains a need for new antitumor agents which have pharmacological activities other than kinase inhibition, since such agents may provide a new treatment option for cancer patients. For example, a new class of antitumor agents which has immunostimulatory effect enhances the immune attack of tumor cells in the body of cancer patients.

Tumor-associated macrophage (TAM) is one of the innate and adaptive immune cells recruited to the tumor site, and thought to generally play a protumoral role. TAM is a potential target of the cancer therapy (Noy et al., Immunity 41:49-61, 2014).

A compound represented by the formula (I), also known as lenvatinib, or a pharmacologically acceptable salt thereof (e.g., lenvatinib mesilate) has been known to have a potent angiogenesis inhibitory effect (U.S. Patent Application Publication No. 2004-0053908) and a kinase inhibitory effect for various receptor tyrosine kinases, including VEGFR (U.S. Patent Application Publication No. 2004-0253205; U.S. Patent Application Publication No. 2009-0209580), and to be useful as a preventive or therapeutic agent against various tumors such as thyroid cancer, hepatocellular carcinoma, endometrial cancer, lung cancer, melanoma, glioblastoma, renal cell carcinoma and ovarian cancer:

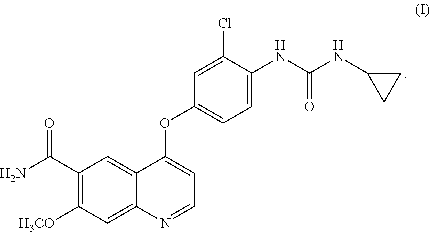 [SMILES: COc1cc2nccc(Oc3ccc(NC(=O)NC4CC4)c(Cl)c3)c2cc1C(N)=O]

### SUMMARY

The present application is based, at least in part, on the identification of novel and unpredicted properties of lenvatinib or a pharmacologically acceptable salt thereof (e.g., lenvatinib mesilate) as an immunostimulant. The present application provides a method of stimulating the immune system of a cancer patient in need thereof, the method comprising administering lenvatinib or pharmacologically acceptable salt thereof to said patient. In one embodiment, such method causes a decrease of the percentage of TAM population in the tumor. In another embodiment, such method causes an activation of cytotoxic T lymphocytes.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A presents the antitumor activities of lenvatinib mesilate in BNL 1ME A.7R.1 murine hepatocellular carcinoma xenografts in athymic.

FIG. 1B presents the antitumor activities of lenvatinib mesilate in BNL 1MEA.7R.1 murine hepatocellular carcinoma xenografts in wild type mice.

Data represent the mean±SD (n=5). Lenvatinib mesilate (3 and 10 mg/kg) was orally administered to the mice once a day for 7 days (Day 1-7).

Lenvatinib=lenvatinib mesilate, Athymic=CAnN.Cg-Foxn1^{nu}/CrlCrlj, Wild-type=BALB/cAnNCrlCrlj.

**** P<0.0001 versus vehicle-treated group on Day 8 (one-way analysis of variance followed by the Dunnett multiple comparison test). There was no significant difference between vehicle- and lenvatinib mesilate-treated groups in Athymic mice on Day 8.

FIG. 2 presents the comparison of antitumor activity of lenvatinib mesilate in BNL 1ME A.7R.1 murine hepatocellular carcinoma xenografts in athymic mice with wild-type mice. Data represent the mean±SD and data from individual mouse (n=5) on Day 8. Lenvatinib mesilate (3 and 10 mg/kg) was orally administered to the mice once a day for 7 days (Day 1-7).

Lenvatinib=lenvatinib mesilate, Athymic=CAnN.Cg-Foxn1^{nu}/CrlCrlj, TGI=tumor growth inhibition, Wild type=BALB/cAnNCrlCrlj.

** P<0.01, *** P<0.001 (unpaired t test).

FIG. 3A presents the effects of lenvatinib mesilate on frequency of immune population (TAM) in tumor of BNL 1MEA.7R.1 murine hepatocellular carcinoma xenografts in wild-type mice.

FIG. 3B presents the effects of lenvatinib mesilate on frequency of immune population (immature TAM) in tumor of BNL 1MEA.7R.1 murine hepatocellular carcinoma xenografts in wild-type mice.

Data represent mean±SD and data from individual wild-type mouse (n=10) on Day 8.

Lenvatinib mesilate (10 mg/kg) was orally administered to the mice once a day for 7 days (Day 1-7). Lenvatinib=lenvatinib mesilate, TAM=tumor-associated macrophage, Wild-type=BALB/cAnNCrlCrlj.

** P<0.01, **** P<0.0001 (unpaired t test).

FIG. 4 presents the effects of lenvatinib mesilate on level of INF-γ⁺ cells per CD8⁺ T cells in dLN of BNL 1MEA.7R.1 murine hepatocellular carcinoma xenografts in wild-type mice.

Data represent the mean±SD and data from dLN in individual mouse (n=10) on Day 8.

Lenvatinib mesilate (10 mg/kg) was orally administered to the wild-type mice once a day for 7 days (Day 1-7).

dLN=draining lymph node, IFN=interferon, Lenvatinib=lenvatinib mesilate, Wild-type=BALB/cAnNCrlCrlj.

*** P<0.001 (unpaired t test).

### DETAILED DESCRIPTION

Lenvatinib or a pharmacologically acceptable salt thereof (e.g., lenvatinib mesilate) as described herein can be synthesized by the methods described U.S. Patent Application Publication No. 2004-0053908.

The term “pharmacologically acceptable salt” as used herein is not particularly restricted as to the type of salt. Examples of such salts include, but are not limited to, inorganic acid addition salts such as hydrochloric acid salt, hydrobromic acid salt, sulfuric acid salt, nitric acid salt or phosphoric acid salt, organic acid addition salts such as acetic acid salt, succinic acid salt, fumaric acid salt, maleic acid salt, tartaric acid salt, citric acid salt, lactic acid salt, stearic acid salt, benzoic acid salt, methanesulfonic acid salt (mesilate), ethanesulfonic acid salt or p-toluenesulfonic acid salt, inorganic base addition salts such as alkaline metal salts such as sodium salt or potassium salt, alkaline earth metal salts such as calcium salt or magnesium salt, aluminum salt or ammonium salt, and organic base addition salts such as diethylamine salt, diethanolamine salt, meglumine salt or N, N-dibenzylethylenediamine salt. It is preferably methanesulfonic acid salt.

There are no particular restrictions on the dosage form of administration of the present invention, and it may usually be orally administered as solid dosage forms such as tablets, capsules, granules, fine granules, lozenge or powder, aqueous forms such as suspensions or solutions, jerry form, or syrup form.

The dosage form may also be parenteral forms such as injection, suppository, ointment or plaster.

An effective amount of lenvatinib or a pharmacologically acceptable salt thereof (e.g., lenvatinib mesilate) of the present invention can suitably be determined by a health care practitioner taking into account, for example, the characteristics of the patient (age, sex, body weight, race, sensitivity to drug, etc.), the progression of the disease, dosage form, route, timing and interval of the administration, and prior exposure to the drug. It will ordinarily be 1 to 600 mg, preferably 5 to 400 mg, more preferably 5 to 200 mg per day, when orally administered to adults (60 kg body weight). It can be administered at once or divided over several times.

The oral dosage forms that can be used for the present invention may be made by adding excipients and, optionally, binders, disintegrants, lubricants, flavoring agents and/or coloring agents to lenvatinib or a pharmacologically acceptable salt thereof (e.g., lenvatinib mesilate), followed by formulating into tablets, capsules, granules, fine granules, lozenges or powder according to techniques known in the art.

The examples of such excipients include, but not limited to, lactose, corn starch, white soft sugar, glucose, sorbitol, microcrystalline cellulose and silicon dioxide.

The examples of such binders include, but not limited to, polyvinyl alcohol, ethylcellulose, methylcellulose, gum arabic, hydroxypropylcellulose and hydroxypropylmethylcellulose.

The examples of such lubricants include, but not limited to, magnesium stearate, talc and silica.

The examples of such flavoring agents include, but not limited to, cocoa powder, ascorbic acid, tartaric acid, Mentha oil, bomeol and powdered cinnamon bark.

The examples of such coloring agents include, but not limited to, titanium oxide, ferric oxide, yellow ferric oxide, cochineal extract, carmine and riboflavin.

The injections that can be used for the present invention may be made by adding acidity regulators, buffers, suspending agents, emulsifiers, solubilizers, stabilizers, tonicity agents and/or preservatives to lenvatinib or a pharmacologically acceptable salt thereof (e.g., lenvatinib mesilate), followed by formulating into suitable forms for intravenous, subcutaneous or intramuscular injection according to techniques known in the art. Such injections may optionally be lyophilized according to techniques known in the art.

The examples of such suspending agents include, but not limited to, methylcellulose, polysorbate 80, hydroxyethyl cellulose, gum arabic, powdered tragacanth, sodium carboxymethyl cellulose and polyoxyethylene sorbitan monolaurate.

The examples of such solubilizers include, but not limited to, polyoxyethylene hydrogenated castor oil, polysorbate 80, nicotinamide, polyoxyethylene sorbitan monolaurate, macrogol and glyceryl fatty acid ester.

The examples of such stabilizers include, but not limited to, sodium sulfite and sodium metabisulfite.

The examples of such preservatives include, but not limited to, methyl-p-hydroxybenzoate, ethyl-p-hydroxybenzoate, sorbic acid, phenol, cresol and chlorocresol.

The term “activation of cytotoxic T lymphocytes” as used herein means a state that cytotoxic T lymphocytes in the patient's body who has been administered lenvatinib or pharmacologically acceptable salt thereof (e.g. lenvatinib mesilate) are activated which can promote an inhibition of the growth or metastasis of the tumor. Such activation may be assayed according to techniques known in the art. For example, such activation may be observed as an increase of IFN-γ secreting CD8⁺ T cells in the tumor or its draining lymph nodes. Alternatively, such activation may be observed as an increase of tumor-specific antigen reactive T cells, which can be assessed by staining of peripheral blood mononuclear cells (PBMCs) with MHC-tetramer, CD3 and CD8, followed by a calculation of the percentage of CD3⁺CD8⁺MHC-tetramer⁺ cells by flow cytometry. Alternatively, the activation of T cells in PBMCs may be detected as an increase of Th1 cytokines such as TNFα, IFN-γ or IL12 or CTL markers such as Perforin or Granzyme B by using an ordinary detection method such as ELISA, ELISPOT or quantitative PCR.

### EXAMPLES

### 1. Materials and Methods

1.1 Test Compound
Name: Lenvatinib mesilate

1.2 Preparation of Test Compound Solutions
Vehicle: Distilled water
Preparation Method: Lenvatinib mesilate was dissolved with the vehicle at a concentration of 1 mg/mL. Aliquot of the 1 mg/mL solution was diluted with the vehicle to yield 0.3 mg/mL. Both solutions were stored at 4° C. until use.

1.3 Cells
Species: Mouse
Tissue: Liver
Designation: BNL 1MEA.7R.1
Supplier: American Type Culture Collection (ATCC)

1.4 Animal
Species/Strain: Mouse/CAnN.Cg-Foxn1^{nu}/CrlCrlj (athymic) and BALB/cAnNCrlCrlj (wild-type)
Sex: Female
Supplier: Charles River Laboratories Japan
Number of Animals Used: Athymic: 25
   Wild-type: 33
Age When Used: 7-week-old

1.5 Antibody List
Target cells of Ex. 1 and 2 were stained with the following antibodies;

| | | | | | | | Final | |
|---|---|---|---|---|---|---|---|---|
| | | | | | | | Conc. | |
| Ex. | Antigen | Fluorescence | Clone | Brand | Isotype | | (mg/mL) | Lot Number |
| 1 | Mouse F4/80 | FITC | BM8 | eBioscience | Rat | IgG2a, κ | 5 | E00611-1634 |
| | Mouse Ly6G(Gr-1) | PE | RB6-8C5 | eBioscience | Rat | IgG2b, κ | 2 | E01925-1632 |
| | Mouse CD45 | PerCP-Cy5.5 | 30-F11 | BD Biosciences | Rat | IgG2b, κ | 2 | 3252618 |
| | Mouse Ly6C | APC-Cy7 | AL-21 | BD Biosciences | Rat | IgM, κ | 2 | 3239513 |
| | Mouse CD11b | PE-Cy7 | M1/70 | BD Biosciences | Rat | IgG2b, κ | 0.5 | 3309921 |
| 2 | Mouse CD3 | Alexa700 | 17A2 | eBioscience | Rat | IgG2b, κ | 2 | E08933-1632 |
| | Mouse CD8a | FITC | 53-6.7 | BD Biosciences | Rat | IgG2a, κ | 2 | 3177592 |
| | Mouse IFN-γ | PE | XMG1.2 | BD Biosciences | Rat | IgG1, κ | 2 | 3217951 |

Conc. = concentration, Ex. = Example

1.6 Study Design

### 1.6.1 Referential Example. 1

### Antitumor Effects of Lenvatinib Mesilate in BNL 1MEA.7R.1 Murine Hepatocellular Carcinoma Cell Line Xenografts in Athymic and Wild-Type Mice

The BNL 1MEA.7R. 1 murine hepatocellular carcinoma cells were cultured in Dulbecco's minimal essential medium (DMEM) supplemented with 10% heat-inactivated (56° C., 30 minutes) fetal bovine serum (FBS) and 100 U/mL penicillin, 100 μg/mL streptomycin, 1-glutamine (2 mmol/L) in humidified atmosphere of 5% CO₂ at 37° C. This tumor cell line was developed from BALB/c mouse. The cells were washed with phosphate buffered saline (PBS), harvested with 0.25% trypsin-EDTA, and suspended with 50% BD Matrigel™ (BD biosciences) in the Hank's balanced salt solution (HBSS) at a density of 1×10⁸ cells/mL. A 0.1 mL aliquot of the cell suspension was inoculated subcutaneously into the right flank of each mouse. Fifty days after the inoculation (Day 1), 15 out of 25 athymic mice and 25 out of 33 wild-type mice were selected based on their tumor volumes, shapes of tumors, physical condition, and body weights. Then the selected athymic and wild-type mice were randomly divided into 3 groups (Group 1-3 in the following table “Treatment Groups”) and 5 groups (Group 4-8), respectively. Animals assigned to Group 4, 6, 7, and 8 were used in Ex. 1 and 2. The mean tumor volumes of athymic and wild-type mice assigned to 8 groups were 169.5 and 157.5 mm³, respectively, on Day 1.

Treatment Groups

| | Type | | | | |
|---|---|---|---|---|---|
| Group | of Mice | Treatment | Ex. | | n |
| 1 | Athymic | Vehicle | Ref. | — | 5 |
| 2 | | Lenvatinib mesilate 3 mg/kg | Ref. | — | 5 |
| 3 | | Lenvatinib mesilate 10 mg/kg | Ref. | — | 5 |
| 4 | Wild-type | Vehicle | Ref. | 1, 2 | 5 |
| 5 | | Lenvatinib mesilate 3 mg/kg | Ref. | — | 5 |
| 6 | | Lenvatinib mesilate 10 mg/kg | Ref. | 1, 2 | 5 |
| 7 | | Vehicle | — | 1, 2 | 5 |
| 8 | | Lenvatinib mesilate 10 mg/kg | — | 1, 2 | 5 |

Athymic = CAnN•Cg-Fox1^{nu}/CrlCrlj, Wild-type = BALB/cAnNCrlCrlj, Ref. = Referential Ex. 1, — = not applicable.

Lenvatinib mesilate (3 and 10 mg/kg) or vehicle was orally administered at 0.1 mL/10 g body weight, once daily for 7 days (Day 1-7). The tumor volume and body weight of mice were measured on Day 1, 3, 6, and 8.

The tumor volume was calculated according to the following formula:

Tumor volume (mm³)=length (mm)×width² (mm²)×1/2

Length: largest diameter of tumor
Width: diameter perpendicular to length

### 1.6.2 Example. 1

### Immunepopulation Analysis in BNL 1MEA.7R.1 Xenografts Tumor of Wild-Type Mice by Flow Cytometory Analysis

Half-cut tumor samples collected from BNL 1MEA.7R.1 xenograft model in wild-type mice 1 day after the administration of vehicle or lenvatinib mesilate (10 mg/kg) in Referential Ex. 1 (Group 4, 6, 7, and 8) were minced with a razor on ice. The tumor fragments were digested with collagenase (1900 U/mL) and deoxyribonuclease I (160 KIU/mL) in HBSS for 1 hour at 37° C. with magnetic stirring in flasks. After filtering through cell strainer (pore size 70 μm), harvested cells were washed 3 times with flow cytometry buffer (PBS supplemented with 0.5% bovine serum albumin and 2 mmol/L EDTA). Cells were resuspended with 200 μL of FCM buffer containing 5 μg/mL Mouse BD Fc Block™ (BD biosciences), and incubated at 4° C. for 10 minutes. Antibody cocktail (dilution; 1:2 in FCM buffer) 1 and 2 were prepared as below.

Composition of Antibody Cocktails

| Antibody Cocktail 1 | | | Antibody Cocktail 2 | | |
|---|---|---|---|---|---|
| | | Final Conc. | | | Final Conc. |
| Antigen | Fluorescence | (μg/mL) | Antigen | Fluorescence | (μg/mL) |
| Mouse F4/80 | FITC | 5 | Mouse CD49b | FITC | 2 |
| Mouse Ly6G(Gr-1) | PE | 2 | Mouse CD8a | PE | 2 |
| Mouse CD45 | PerCP-Cy5.5 | 2 | Mouse CD45 | PerCP-Cy5.5 | 2 |
| Mouse Ly6C | APC-Cy7 | 2 | Mouse CD3 | Alexa700 | 2 |
| Mouse CD11b | PE-Cy7 | 0.5 | Mouse CD11b | PE-Cy7 | 0.5 |
| — | DAPI | 10 | — | DAPI | 10 |

Conc. = concentration, — = not applicable.

One hundred microliters of cell suspension was mixed with 100 μL of the antibody cocktail 1 or 2 in each well of two 96-well cell culture round bottom plates (Corning Incorporated), and incubated at 4° C. for 30 minutes in a light protected. After centrifugation at 1200 rpm at 4° C. for 5 minutes, the supernatant was discarded and the resultant cells were washed 3 times with FCM buffer, and resuspended with 200 μL/well of FCM buffer. FCM analysis was performed by FACSAria II SORP cell sorter (Becton, Dickinson and company). Data were analyzed by FCM analysis software FlowJo 7.6.5 (Tree Star, Inc.).

### 1.6.3 Example. 2

### IFN-γ Staining Analysis of CD8⁺ T Cells Derived from Draining Lymph Nodes in BNL 1MEA.7R.1 Xenografts in Wild-Type Mice

Draining lymph nodes derived from BNL 1MEA.7R. 1 xenograft model in wild-type mice 1 day after once-daily oral administration of vehicle or lenvatinib mesilate (10 mg/kg) for 7 days in Referential Ex. 1 were isolated and mashed by a 5-mL syringe. The cell number in suspension was calculated and adjusted to 2.0×10⁶ cells/mL with RPMI1640 containing 10% FBS, 100 U/mL penicillin, and 100 μg/mL streptomycin. One hundred microliters per well of the resultant cell suspension were plated in a 96-well cell culture cluster round bottom plate (Corning Incorporated) and cultured with BD Goldi-Stop™ (final concentration [conc.] 4 μL/6 mL) for 5 hours in 5% CO₂ at 37° C. After centrifugation at 1200 rpm at 4° C. for 2 minutes, supernatant was discarded and the resultant cells were incubated with 100 μL of PBS with 2% FBS containing 1 μL of mouse BD Fc block in each well for 10 minutes on ice. After centrifugation at 1200 rpm at 4° C. for 2 minutes, supernatant was discarded. Then 50 μL/well of a mixture of Alexa700-labeled anti-CD3 antibody (final conc. 2 μg/mL) and FITC-labeled anti-CD8a antibody (final conc. 2 μg/mL) was added into the cells and they were incubated on ice for 20 minutes. After washing the cells by adding PBS with 2% FBS, 100 μL of BD Fixation/Permeabilization solution (a kit component of BD Cytofix/Cytoperm™ Fixation/Permeabilization kit with BD GolgiStop™) was added in each well and incubated for 20 minutes at room temperature. After washing the cells with BD Perm/Wash buffer which was a component of the kit, the resultant cells were cultured with 50 μL/well of PE-labeled anti-IFN-γ antibody solution (final conc. 2 μg/mL) for 20 minutes at room temperature. After washing with 200 μL of BD Perm/Wash buffer 2 times, the resultant cells were resuspended with 200 μL of BD Perm/Wash buffer, and analyzed to detect percentage of IFN-γ-positive cells in CD3- and CD8-positive cells by FACSAriaII SORP cell sorter (Becton, Dickinson and company) with BD FACSDiva 6.1.3 software (Becton, Dickinson and company). All incubation for immunofluorescent staining were conducted in a light protected.

1.7 Statistical Analysis

### Referential Ex. 1

Data are expressed as mean±SD. The differences in relative tumor volume on Day 8 between vehicle- and lenvatinib mesilate-treated groups (3 and 10 mg/kg) in athymic and wild-type mice were analyzed by one-way analysis of variance followed by the Dunnett multiple comparison test, respectively.

The percentage of TGI was calculated according to the following formula;

% TGI=(1−dT/dC)×100

dT=Tumor volume on Day 8−Tumor volume on Day 1
dC=Mean tumor volume of the vehicle-treated group on Day 8−Mean tumor volume of the vehicle-treated group on Day 1

To compare antitumor activities of lenvatinib mesilate between different strains, differences in % TGI of lenvatinib mesilate between athymic and wild-type mice at each dose were analyzed by the unpaired t test.

A value of P<0.05 (two sided) was considered statistically significant. Statistical analyses were performed using GraphPad Prism version 6.02 (GraphPad Software, Inc.).

### Ex. 1

Data are expressed as mean±SD. The differences in percentage of target cells in CD45⁺ cells between vehicle- and lenvatinib mesilate-treated groups were analyzed by the unpaired t test.

A value of P<0.05 (two sided) was considered statistically significant. Statistical analyses were performed using GraphPad Prism version 6.02.

### Ex. 2

Data are expressed as mean±SD. The differences in percentage of IFN-γ-positive cells in CD8⁺ T cells between vehicle- and lenvatinib mesilate-treated groups were analyzed by the unpaired t test

A value of P<0.05 (two sided) was considered statistically significant. Statistical analyses were performed using GraphPad Prism version 6.02.

### 2. Results

### Referential Ex. 1

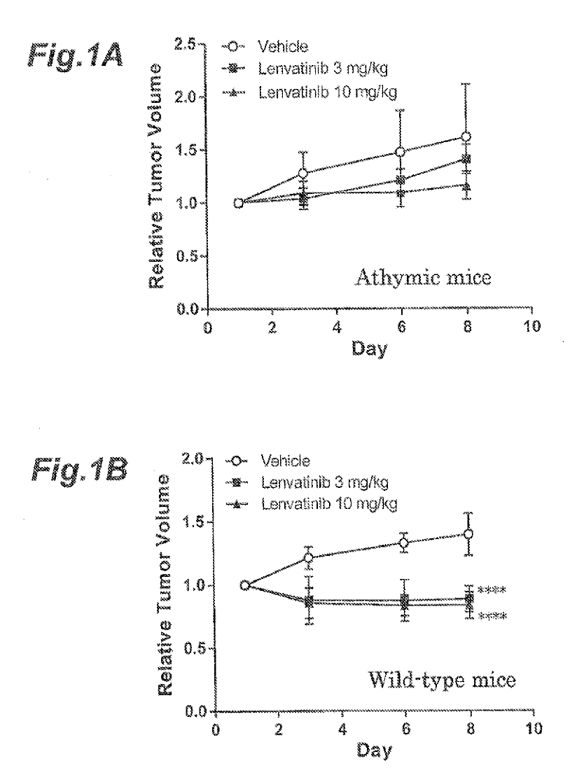

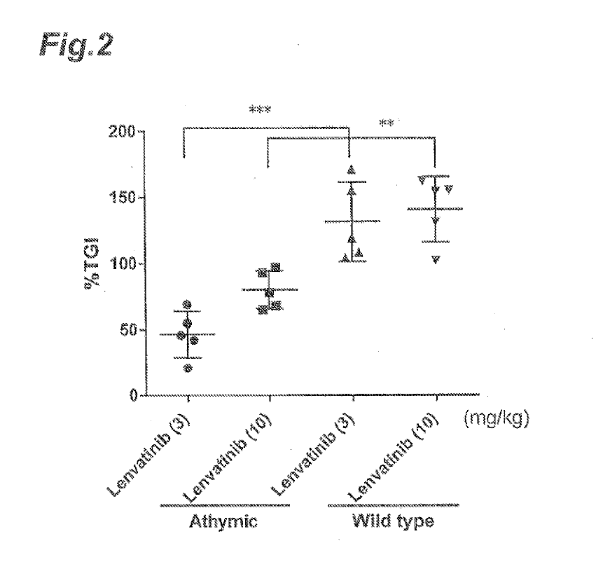

Oral lenvatinib mesilate at 3 and 10 mg/kg showed a significant reduction in tumor volume compared to that of vehicle in BNL 1MEA.7R.1 xenografts on Day 8 in wild-type mice (FIG. 1B), although it showed only tumor growth delay in athymic mice (FIG. 1A). The % TGI of lenvatinib mesilate at both 3 and 10 mg/kg in wild-type mice were significantly larger than those at each dose in athymic mice (FIG. 2). Any severe body weight loss was not observed during the study period.

### Ex. 1

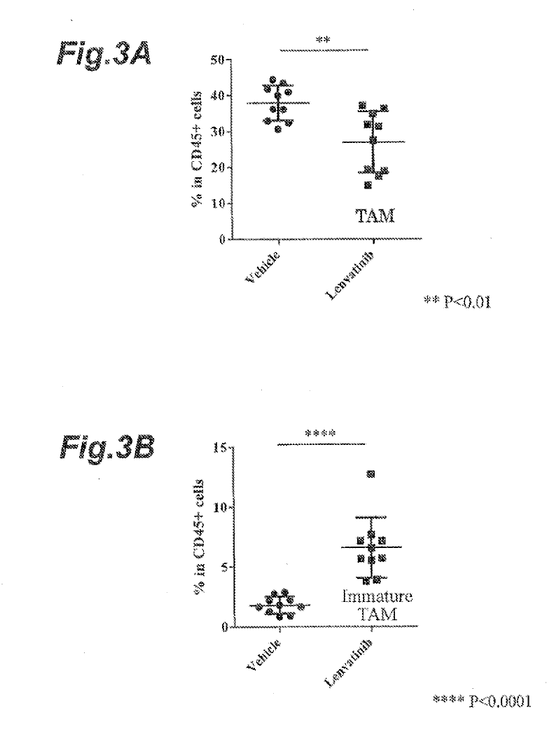

Oral treatment of lenvatinib mesilate at 10 mg/kg once a day for 7 days showed a significant reduction of percentage of TAM population (CD45⁺CD11b⁺Ly6G⁻F4/80⁺Ly6C^{low}) in total CD45⁺ cells in the sample, compared to vehicle treatment (FIG. 3A), although it increased that of immature TAM (CD45⁺CD11b⁺Ly6G⁻F4/80⁻Ly6C^{high}) (FIG. 3B).

### Ex. 2

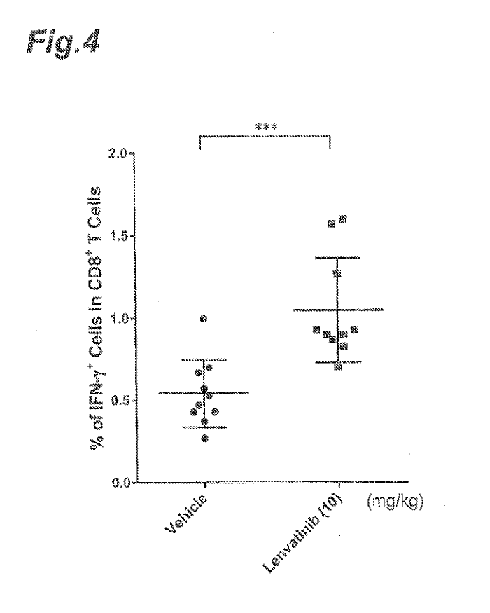

Oral treatment of lenvatinib mesilate at 10 mg/kg once a day for 7 days significantly increased percentage of IFN-γ⁺ cells in CD8⁺ T cells in dLN of wild-type mice inoculated with BNL 1 MEA.7R. 1 cells, compared to vehicle treatment (FIG. 4).

### 3. Conclusion

Compared to athymic mice, increases in tumor volume of BNL 1MEA.7R.1 murine hepatocellular carcinoma cell line xenograft model in wild-type mice significantly inhibited by once-daily oral administration of lenvatinib mesilate at the doses of 3 and 10 mg/kg for 7 days. In both treatment groups, tumors from all animals shrank on Day 8. In addition, % TGIs of lenvatinib mesilate at both 3 and 10 mg/kg in wild-type mice showed significantly higher tumor growth inhibition than those of athymic mice in this study. Administration of lenvatinib mesilate at 10 mg/kg for 7 days significantly decreased percentage of TAM population (CD45⁺CD11b⁺Ly6G⁻F4/80⁻Ly6^{low}), although it increased percentage of immature TAM population (CD45⁺CD11b⁺Ly6G⁻F4/80⁻Ly6C^{high}). Furthermore, activated CD8⁺ T cells population (IFN-γ⁺CD3⁺CD8⁺) was increased in dLN by administration of lenvatinib mesilate at 10 mg/kg. All of these results showed that lenvatinib mesilate has immunostimulatory effect by decreasing TAM.

What is claimed is:

1. A method of stimulating the immune system of a cancer patient in need thereof, the method comprising administering lenvatinib or a pharmacologically acceptable salt thereof to said patient.

2. The method of claim 1, wherein the method causes a decrease of the percentage of tumor-associated macrophage (TAM) population in the tumor.

3. The method of claim 1, wherein the method causes an activation of cytotoxic T lymphocytes.

4. The method of claim 3, wherein the activation of cytotoxic T lymphocytes is observed as an increase of IFN-γ secreting CD8⁺ T cells in the tumor or its draining lymph nodes.

5. The method of claim 1, wherein the lenvatinib or pharmacologically acceptable salt thereof is lenvatinib mesilate.

6. The method of claim 2, wherein the lenvatinib or pharmacologically acceptable salt thereof is lenvatinib mesilate.

7. The method of claim 3, wherein the lenvatinib or pharmacologically acceptable salt thereof is lenvatinib mesilate.

8. The method of claim 4, wherein the lenvatinib or pharmacologically acceptable salt thereof is lenvatinib mesilate.
